(19) United States
(12) Patent Application Publication
(10) Pub. No.: US 2006/0281165 A1
(43) Pub. Date: Dec. 14, 2006

(54) Method for stabilization of enzymes during exposure to sterilizing radation

(76) Inventors: Paul James Davis, Bedford (GB); Andrew John Austin, Northants (GB)

Correspondence Address: MORGAN LEWIS & BOCKIUS LLP, 1111 PENNSYLVANIA AVENUE NW, WASHINGTON, DC, 20004, US

(21) Appl. No.: 10/557,991

(22) PCT Filed: Jun. 4, 2004
(86) PCT No.: PCT/GB04/02392

§ 371 Date: Nov. 22, 2005

(30) Foreign Application Priority Data
Jun. 9, 2003 (GB) 0313217.2
Jan. 30, 2004 (EP) 04250509.9

(51) Int. Cl. C12N 9/94

(52) U.S. Cl. 435/186

(57) ABSTRACT

The present invention provides a composition comprising an enzyme, e.g. oxidase enzyme, a source of zinc and/or ammonium ions and a source of lactate ions. The compositions are typically sterilised by exposing the compositions to sterilising radiation, e.g. gamma radiation. The incorporation of a source of zinc and/or ammonium ions and a source of lactate ions, e.g. zinc L-lactate, in the enzyme-containing composition results in an improvement in enzyme activity post-sterilisation. The presence of a source of zinc and/or ammonium ions and a source of lactate ions in the composition therefore has a protective effect on the enzyme during exposure to sterilising radiation so that good recovery of enzyme activity can be obtained.

### FIELD OF THE INVENTION

This invention relates to compositions comprising enzyme, e.g. an oxidase enzyme, to products including such compositions and to a method of stabilising an enzyme in a composition.

### BACKGROUND TO THE INVENTION

Enzymes that have been extracted and prepared for use in artificial applications as dilute aqueous solutions (e.g. at concentrations ranging from 1 μg/ml to 10 mg/ml) are usually unstable, and it is normal practice to store them at about 4° C., or to freeze them. It is also known to maintain enzymes in a stable, active condition by keeping them dry, e.g. by freeze-drying (lyophilisation) or by drying them in a sugar “glaze” (sugar vitrification). Alternatively, they may sometimes be made stable by precipitation in a saturated solution of ammonium sulphate (in which state they would also normally be kept at a low temperature). Enzymes that are dissolved in water are usually kept in an active condition by keeping them refrigerated or frozen at relatively high concentrations.

Another approach is to use additives to stabilise enzymes. Many compositions have been described in the literature, in which enzymes in solution are mixed with additives that in some way prevent degradation of the enzyme, e.g. by heat or chemicals. Examples of suitable additives known to stabilise enzymes include mixtures of polyelectrolytes such as poly(methyl vinyl ether-co-maleic anhydride) (known as Gantrez where Gantrez is a Trade Mark), polysaccharides such as dextran sulphate, and neutral water-soluble polymers such as polyvinyl pyrrolidone. Many of these additives work best when the enzyme in the resulting composition is to be freeze-dried or stored at high concentrations prior to being diluted to working strength. Very few additives are known to be efficacious in the stabilisation of enzymes whilst they are dissolved in water or aqueous solutions, or are in intimate contact with water, e.g. fixed to a wet surface or coupled to a dissolved polymer or gel-forming polymer in an aqueous medium, particularly when the enzymes are present at working strength, i.e. in dilute form.

It can therefore be difficult not only to stabilise enzymes in certain circumstances but also to maintain their activity through the various stages of preparing an enzyme-containing composition, particularly in the form of a product, so that the enzyme is active in use of the composition. This is particularly but not exclusively so when the enzyme is in hydrated condition.

These difficulties are generally compounded when the composition incorporating the enzyme needs to be sterile. That is, the composition requires sterilisation by exposure to radiation to exclude unwanted bacteria.

It is known that enzymes in contact with water are easily damaged when exposed to various types of radiation. Those skilled in the art therefore appreciate that use of radiation should be avoided when enzymes are present in, e.g. hydrated condition, in a composition, particularly if those enzymes need to be intact and undamaged in use of the composition.

Sterilisation by irradiation is a particularly aggressive process requiring a composition to typically be subjected to high doses of sterilising radiation, generally in the region of 25 to 40 kGy. These conditions are especially damaging to enzymes present in a composition at generally dilute working strength and/or to enzymes which are not immobilised in a composition, e.g. by being irreversibly bound to a solid support.

The present invention aims to address such stability problems.

### SUMMARY OF THE INVENTION

In one aspect the present invention provides a composition comprising an enzyme, a source of lactate ions and a source of zinc ions and/or a source of ammonium ions.

The present invention is based on the observation that when compositions including an enzyme, particularly but not exclusively in hydrated condition, are exposed to sterilising radiation, enzyme activity from the resulting sterilised composition is typically poor. Exposing the composition to sterilising radiation gives rise, in general, to an almost complete loss of enzyme activity following irradiation. It has now however been appreciated that the incorporation of a source of zinc and/or ammonium ions and a source of lactate ions in the enzyme-containing composition results in an improvement in enzyme activity post-sterilisation. The presence of a source of zinc or ammonium ions and a source of lactate ions in the composition therefore appears to have a protective effect on the enzyme during exposure to sterilising radiation so that good recovery of enzyme activity can be obtained.

The effect of zinc ions is particularly surprising in view of the recent findings of Akhtar, M S et al (2002) Biochemistry 41, 7142-7149 that, even at low concentrations, divalent cations cause unfolding of glucose oxidase and inhibition of enzyme activity.

The invention finds application with a wide range of enzymes, including oxidase enzymes, particularly oxidoreductase enzymes, catalase and lactoperoxidase.

Glucose oxidase is an example of an oxidase enzyme which catalyses the reaction of an appropriate substrate with oxygen to produce hydrogen peroxide (H₂O₂), a known antimicrobial substance with many advantages. Hydrogen peroxide is produced naturally in the body by white blood cells as part of the immune defence activities in response to infection. There are no known adaptive microbial evasion mechanisms by which microbes can escape its effects and it has a short lifetime, very rapidly breaking down to water and oxygen in the tissues. It therefore does not accumulate to dangerous levels. When it is to be applied topically (e.g. to treat acne), its effectiveness is enhanced by the fact that it readily penetrates the skin surface to reach underlying sites of infection. As hydrogen peroxide is so beneficial, it has been used for many years as a biologically compatible general antiseptic and as an antimicrobial substance for cleansing wounds of all kinds.

Moreover, hydrogen peroxide can be used as a substance with which to transport oxygen through a water-bearing dressing, by virtue of its solubility in the water of the dressing, and its ready decomposition by catalase present naturally within the wound, or in the tissue surrounding the wound.

The enzyme may be in hydrated condition or dry condition in the composition. When the enzyme is in hydrated condition, the enzyme is present in a wet, active state in the composition and can begin functioning immediately when brought into contact with appropriate substrate on use of the composition. Alternatively, the enzyme may be in dry condition and require initial hydration on use. Preferably, the enzyme is present in hydrated condition in the composition.

Oxidase enzymes suitable for use in compositions of the invention and the corresponding substrates (which, e.g. may be present in bodily fluids such as blood and tissue fluids and/or be supplied separately to prevent premature oxidation reaction and/or be incorporated in the composition) include the following:

| Enzyme | Substrate |
|---|---|
| Glucose oxidase | β-D-glucose |
| Hexose oxidase | Hexose |
| Cholesterol oxidase | Cholesterol |
| Galactose oxidase | D-galactose |
| Pyranose oxidase | Pyranose sugars |
| Choline oxidase | Choline |
| Pyruvate oxidase | Pyruvate |
| Glycollate oxidase | Glycollate |
| Aminoacid oxidase | Aminoacid |

The currently preferred oxidase enzyme is glucose oxidase. This catalyses reaction of β-D-glucose substrate to give hydrogen peroxide and gluconic acid.

A mixture of oxidase enzymes may be used.

Appropriate amounts of oxidase enzyme, or other enzyme, for a particular composition can be readily determined by experiment.

The source of zinc ions may be any compound capable of releasing zinc ions or zinc-containing ions in water. Suitable sources of zinc ions include, for example, zinc chloride, zinc fluoride, and zinc sulphate.

The source of ammonium ions may be any compound capable of releasing ammonium ions or ammonium-containing ions in water. Suitable sources of ammonium ions include ammonium sulphate and 2-acrylamido-2-methyl propanesulphonic acid, ammonium salt (ammonium AMPS) (e.g. available under the Trade Mark Lubrizol AMPS 2411 Monomer), the latter conveniently being a monomer material of a polymer hydrogel.

The source of lactate ions may be any compound capable of releasing lactate ions or lactate-containing ions in water. The lactate ion (derived from lactic acid) is optically active and so may exist in two enantiomeric forms, L- and D-, and as a mixture of both enantiomers, known as a racemate. Any enantiomeric form, or any mixture of enantiomeric forms, is suitable for use herein. Convenient sources of lactate ions include sodium L-lactate, sodium D-lactate, sodium D, L-lactate and zinc L-lactate, although it is believed that any soluble lactate can be used as a source of lactate ions.

Experiments with oxidase enzymes have shown that good levels of enzyme activity are retained following exposure to sterilising radiation for compositions containing a source of zinc ions and a source of lactate ions.

A currently preferred source of zinc ions and lactate ions is zinc lactate, particularly zinc L-lactate. In a composition of the invention, zinc L-lactate has been shown to have a stabilising effect on the enzyme when present at a concentration of at least 0.2% by weight based on the total weight of the composition. Increasing the concentration of zinc L-lactate in the composition results in an increase in the stabilising effect. Particularly good results may be obtained when zinc L-lactate is present at a concentration of about 1.0% by weight based on the total weight of the composition. Solubility problems can however arise when zinc L-lactate is employed in a composition at higher concentrations.

Experiments with oxidase enzymes have shown that good levels of enzyme activity are retained following exposure to sterilising radiation for compositions containing a source of ammonium ions and a source of lactate ions. In this case, better results are obtained using sodium lactate than zinc lactate (weight for weight), so sodium lactate is preferred in this situation. Further, higher concentrations of sodium lactate can be used compared with zinc lactate, as sodium lactate is more soluble in water. Sodium lactate is conveniently present in the composition at a concentration of about 1.0% by weight or more, e.g. up to about 4.0% by weight, based on the total weight of the composition. Good results have been obtained with compositions containing oxidase enzyme, ammonium AMPS (e.g. as the monomer material of a polymer hydrogel) and sodium lactate.

Further experiments with catalase and lactoperoxidase enzymes have shown that good levels of enzyme activity are retained following exposure to sterilising radiation for compositions containing a source of lactate ions and a source of zinc ions (e.g. zinc lactate), and additionally containing a source of ammonium ions (e.g. ammonium AMPS) in the case of lactoperoxidase.

In a composition of the invention, ammonium ions may be present at a concentration of at least 0.5%, preferably at least 1%, and more preferably at least 2%, by weight based on the total weight of the composition. In such a composition, in which ammonium ions are present, it is possible to omit zinc ions without compromising the stabilisation effect. In this case, the lactate ions can be added, e.g. in the form of sodium lactate, at a concentration up to 4% by weight.

Compositions in accordance with the invention may optionally include other ingredients known to stabilise enzymes (hereinafter for brevity and simplicity referred to as “stabilisers”).

Suitable known stabilisers for use herein include sugar alcohols such as mannitol, sorbitol, xylitol and lactitol; proteins such as gelatin; and neutral water-soluble polymers such as polyvinyl pyrrolidone and polyvinyl alcohol (e.g. having a molecular weight in the range of about 30,000 to 100,000).

Sugar alcohols can typically be used in compositions in accordance with the invention at concentrations in the range 0.5% to 4% by weight based on the total weight of the composition.

If proteins are employed, they may be present in compositions of the invention at a concentration of at least 0.5%, preferably at least 1%, and more preferably at least 4%, by weight based on the total weight of the composition.

Neutral water-soluble polymers can be used with good effect typically at concentrations in the range of 0.5% to 3.5% by weight based on the total weight of the composition.

The stabilising effect of lactate ions in concert with zinc or ammonium ions can be enhanced by use of one or more of the above-mentioned optional ingredients. Particularly preferred ingredients for use herein are a source of proteins, especially gelatin.

Compositions in accordance with the invention may conveniently be sterilised by irradiating the composition with sterilising radiation, typically in the region of 25 to 40 kGy. It will therefore be appreciated that the invention also covers a sterilised composition having the features described above. Compositions may be sterilised by exposure to, for example, gamma radiation, x-ray radiation, or electron beam radiation. Usually, compositions are irradiated with gamma radiation.

The presence of zinc and/or ammonium ions and lactate ions in enzyme-containing compositions, enables the compositions to be exposed to sterilising radiation with considerably reduced loss of enzyme activity than would be the case without the lactate ions and the zinc and/or ammonium ions. In particular, enzymes can retain levels of activity that are practically useful and economically acceptable.

Thus, in a further aspect the present invention relates to a method of stabilising an enzyme in a composition during exposure to sterilising radiation by bringing the enzyme into contact with a source of zinc ions and/or ammonium ions and a source of lactate ions.

The compositions of the present invention may be used in a wide range of products for a variety of purposes. Thus, in an even further aspect, the present invention provides a product comprising a composition in accordance with the invention.

Currently favoured products containing an oxidase enzyme are suitable for use on, or application to, the skin, and are in particular skin treatment products. Such products may be formulated in various product forms, e.g. as lotions, creams, gels, sticks or dressings. Preferred skin treatment products comprising a composition in accordance with the invention are skin dressings, particularly wound dressings. Skin dressings may be used by being applied to or located on the skin of a human or animal, e.g. over a wound or on a region of skin to be treated for cosmetic or therapeutic purposes, e.g. for treatment of acne or other skin conditions. The dressings may be generally formulated as described in International Patent Application No. PCT/GB03/01738, published as WO 03/090800.

In a preferred embodiment, the dressing includes one or more water-based or aqueous gels, also referred to as hydrated hydrogels. A hydrated hydrogel provides a source of water so the enzyme is maintained in hydrated condition in such dressings, promoting rapid reaction and consequent release of an antimicrobial substance or oxygen transport.

The or each hydrated hydrogel conveniently comprises hydrophilic polymer material. Suitable hydrophilic polymer materials are as described in WO 03/090800, and mixtures of hydrophilic polymer materials may be used in a gel. In a hydrated hydrogel of hydrophilic polymer material, the hydrophilic polymer material is desirably present at a concentration of at least 1%, preferably at least 5%, more preferably at least 30%, possibly 40%, by weight based on the total weight of the gel.

In particular, the enzyme, source of zinc and/or ammonium ions and source of lactate ions may be present in one or more hydrated hydrogels.

The dressing may also include a further hydrated hydrogel (e.g. of poly AMPS), containing no enzyme but possibly containing substrate for the oxidase enzyme (e.g. a source of glucose for glucose oxidase), additionally or alternatively containing a supply of iodide ions (e.g. in the form of one or more iodide salts) and optionally also containing glycerol.

The or each gel may be cross-linked as described in WO 03/090800.

The hydrated hydrogel, particularly a cross-linked gel, may be cast around a mechanical reinforcing structure, such as a sheet of cotton gauze or an inert flexible mesh, e.g. to providing a structurally reinforced hydrogel layer or slab.

The hydrated hydrogel may alternatively be in the form of a non-cross-linked shear-thinning gel, e.g. of suitable gums such as xanthan gum (e.g. available under the Trade Mark Keltrol), in this case preferably without a mechanical reinforcing structure. Such gums are liquid when subjected to shear stress (e.g. when being poured or squeezed through a nozzle) but set when static. Thus the gel may be in the form of a pourable component, facilitating production of gels in the dressing. Such a shear-thinning gel may also be used in combination with a preformed, mechanically reinforced gel, as discussed above.

The water-absorbing gel may utilise an increased concentration of hydrophilic substance, which may be the actual gel-forming polymer material, e.g. polysaccharide, itself or an additional substance added into the mixture for the sole purpose of absorbing water. One example of this type of functional mixture is that formed by a combination of cross-linked alginate at about 2% by weight and xanthan gum at about 5-10% by weight, based on the total weight of the gel. A particularly favoured version is that of covalently linked polymeric hydrogel such as polyAMPS, which is strongly water absorbing, being able to take up very large volumes of water or aqueous solutions, which is helpful when the skin dressing is used over a wound.

The enzyme (or enzymes) may be present in a gel in a number of possible forms, including in solution as free molecules. To improve efficiency of retention of the enzyme in the gel, the enzyme may be chemically conjugated to another enzyme, chemically conjugated to other molecules (e.g. polyethylene imine), or incorporated in a solid support such as beads.

Gels of different types, e.g. cross-linked alginate and shear-thinning, may be used together in a single dressing. Good results have been obtained with a shear-thinning gel nearest the skin, in use, and a cross-linked structurally reinforced gel remote from the skin.

The enzyme may be immobilised so it can be prevented from being released, e.g. into the blood circulation, where it would have the potential to trigger undesirable allergic responses (being generally derived from non-human sources, e.g. with most commercially available glucose oxidase being derived from the fungus Aspergillius niger) and would also be susceptible to degradation by the effect of proteases present in a wound. An enzyme may be immobilised as described in WO 03/090800.

The dressing desirably includes a source of substrate for the oxidase enzyme, e.g. glucose for glucose oxidase. Preferably the glucose is in the form of pure, pharmaceutical grade material. Glucose can also be supplied in the form of honey which naturally provides other benefits such as healing and antimicrobial factors. The substrate is desirably physically separated from the oxidase enzyme as described in WO 03/090800 prior to use of the dressing, to prevent premature reaction, although because oxygen is required for reaction then provided the supply of oxygen is limited only little reaction can occur. The substrate, e.g. glucose is typically present in an amount up to about 25%, e.g. about 5%, by weight of the weight of the dressing. The substrate, e.g. glucose, may be present in various forms including dissolved within a hydrated hydrogel structure, present as a slowly dissolving solid, or encapsulated within another structure for slow release.

It is helpful to balance the relative amounts of enzyme and substrate such that there is an excess of hydrogen peroxide.

Preferably, the enzyme and substrate are located in separated hydrated hydrogels, with the oxidase enzyme, source of zinc and/or ammonium ions and source of lactate ions located in a first hydrated gel and the substrate located in a second hydrated gel. The dressing desirably has a layered, stratified construction, e.g. comprising an upper (outer) layer of one gel and a lower (inner) layer of another gel. For example, the first gel (with oxidase enzyme, source of zinc ions and source of lactate ions) may be located in the vicinity of the outer parts of the dressing, i.e. remote from the skin in use, where oxygen levels are highest, with the second gel (with substrate) being located in the vicinity of the inner parts of the dressing, i.e. adjacent the skin in use. In this case the dressing thus has a layered, stratified construction, comprising an upper (outer) layer of the first gel and a lower (inner) layer of the second gel.

The antimicrobial efficiency of the system can be further enhanced by the inclusion of iodide ions, which can be oxidised to elemental iodine (which is a known powerful antimicrobial agent, e.g. as discussed in WO 01/28600) by the action of hydrogen peroxide, with or without catalytic effect. Thus, the dressing desirably includes a supply of iodide ions, e.g. potassium iodide or sodium iodide. The supply of iodide ions, e.g. iodide salt, may be provided in a relatively quick-release form, either in the substrate gel or in an additional membrane or gauze or other suitable layer. The supply of iodide may alternatively be located with the source of substrate for the oxidase enzyme, as discussed above, e.g. in a hydrated gel. The iodide may be present in various forms, including dissolved within a hydrated gel structure, present as a slowly dissolving solid, or encapsulated within another structure for slow release. Iodide salt may be present, e.g. in an amount up to about 2% by weight.

The dressing may also include oxygen permeable secondary dressings, such as Tegaderm from 3M Healthcare Ltd or OpSite from Smith & Nephew (Tegaderm and OpSite are Trade Marks), which are made from thin polyurethane film coated on one side with an oxygen permeable adhesive layer.

Such secondary dressings may have features as described in WO 03/090800. Optionally the covering includes a window (or further window) in or through which can be seen indicator means e.g. an indicator sheet or similar structure that indicates (e.g. by changing colour) when the dressing chemistry is active.

One or more components of the dressing may be contained within an enclosure such as a sachet or bag of barrier material that is permeable to oxygen, water and hydrogen peroxide but that prevents undesired migration of materials. Suitable barrier material are described in WO 03/090800.

The water-absorbing components of the dressing can easily be applied to the wound or site of infection, especially when formulated into a workable or flowable form. Suitable arrangements for applying such formulations are described in WO 03/090800.

Dressings of layered construction comprising shear-thinning gels can be readily produced, e.g. by an end user, by pouring or dropping the gels one on top of the other in appropriate order to produce a desired layered assembly of gels. Thus the different dressing component gels may be supplied in separate containers e.g. tubes or bottles or possibly a multi-compartment jar. The different gels may be colour-coded with appropriately coloured latex, for example, to allow ease of identification. The gels may be applied directly to the skin of a user. A covering or outer layer may not be required with such embodiments.

Dressings (or components thereof) are suitably supplied in sterile, sealed, water-impervious packages, e.g. laminated aluminium foil pouches.

Dressings can be manufactured in a range of different sizes and shapes for treatment of areas of skin, e.g. wounds of different sizes and shapes.

The invention is described, by way of illustration, in the following examples.

The ensuing examples describe the effect of a number of ingredients (including zinc L-lactate) on the activity of glucose oxidase, catalase and lactoperoxidase when sterilised in hydrated state in a composition by gamma irradiation using the typical conditions for microbial sterilisation. The effect of additional ingredients capable of augmenting the underlying stabilization provided by lactate ions is also described. The effect was studied using poly-AMPS hydrogels containing glucose oxidase, as well as simple aqueous solutions.

Hydrogels were prepared by firstly dissolving all of the ingredients, including monomers and cross linkers, to form an aqueous liquid which was then converted into a hydrogel slab by exposure to intense UV illumination. The hydrated hydrogel slabs were then irradiated with gamma rays (by an industry-standard sterilizing service), using a dose range as normally specified for hydrogel slabs composed of poly-AMPS. Finally, the gamma-irradiated hydrogel slabs were analysed for remaining enzyme activity. Simple aqueous solutions were prepared as described in the relevant examples.

TABLE 1
Composition of hydrogels used in the study.

| | | |
|---|---|---|
| | Concentration | Concentration |
| | of the stock | in the final |
| Ingredient (supplier) | solution (w/w) | gel (w/w) |

Core Ingredients (common to all
hydrogels used in the study)

| | | |
|---|---|---|
| Sodium AMPS¹ (ex Lubrizol, | 50% aq | 30% |
| code 2405) | | (or 15% if |
| | | ammonium |
| | | AMPS |
| | | also present) |
| Ebecryl 11² (ex UCB Chemicals, | undiluted | 0.20% |
| a cross-linker) | | |
| 1-Hydroxycyclohexyl phenyl | undiluted | 0.01% |
| ketone 99% (ex Aldrich, code | | |
| 40, 561-2, a photoinitiator) | | |
| Glucose oxidase (ex Biocatalysts- | solid powder | 350 ug/g |
| G638P) | | |
| Water (ex Fisher, distilled, | | to total weight |
| deionised, analytical grade) | | |
| Optional Ingredients | | |
| Ammonium AMPS³ (ex Lubrizol, | 50% aq | 15% |
| code 2411) | | |
| Zinc L-lactate (hydrate, ex Aldrich) | 5% aq | 0.2% or 1% |
| Magnesium chloride (ex BDH) | 20% aq | 4% |
| Sodium chloride (ex Fisher) | 20% aq | 4% |
| Calcium chloride (ex Fisher) | 40% aq | 5% |
| Potassium phosphate (ex Fisher) | 10% aq | 2% |
| Ammonium sulphate (ex Fisher) | 20% aq | 4% |
| Gelatin (ex Sigma) | 20% aq | 0.5% to 3.5% |
| PVA⁴ (MW either 31K-50K or | 5% aq | 1% |
| 89K-90K) (ex Aldrich) | | |
| Zinc chloride (ex Sigma) | 20% aq | 2% |
| Zinc sulphate (ex Sigma) | 20% aq | 1% or 2% or 4% |
| Sodium lactate (ex Fisher) | 10% aq | 1% |
| Glycerol (ex Fisher) | 50% aq | 10% |

¹= 2-acrylamido-2-methylpropanesulfonic acid, sodium salt
²= PEG 400 diacrylate
³= 2-acrylamido-2-methylpropanesulfonic acid, ammonium salt
⁴= polyvinyl alcohol

Preparation of Hydrogels

Hydrogels were prepared from ingredients set out in Table 1 above. The particular ingredients were mixed in the quantities illustrated in this table, following the basic procedure below:

Stock solutions (as supplied by the manufacturer) of sodium AMPS and/or ammonium AMPS were dispensed into a 125 ml polypropylene, screw-top reaction jar as the basis of the pre-gel fluid. Glucose oxidase and the optional ingredient(s) (if required) were added to the mixture and allowed to dissolve completely. In a separate vessel 1-hydroxycyclohexyl phenyl ketone was dispersed in the liquid Ebecryl 11 and the mixture was warmed gently (up to 45° C.) to dissolve the photoinitiator into the cross-linker. This solution was then mixed into the pre-gel fluid. To cast the gels, the complete pre-gel fluid was poured into a flat bottomed tray, to a depth of 1-2 mm. The gels were set by UV irradiation from a 1 KW lamp, at a vertical distance of 15 cm, for 25 seconds. The hydrogels were allowed to cool before use. The hydrogels were assayed using the procedure described below before being exposed to gamma irradiation for 25 seconds to give a radiation dose of approximately 100 mWcm⁻¹.

Gamma Irradiation

The hydrogels were gamma irradiated by means of an industry-standard, commercial sterilizing service, provided by Isotron PLC. Using Cobalt 60 source, the dose range in this standard procedure is guaranteed to be in the range of 25-40 kGy, although the gels used in this study were found to have received a dose of approximately 30 kGy in all cases.

Glucose Oxidase Activity Assay

The hydrogel slabs, both pre- and post-gamma irradiated, were assayed for glucose oxidase activity. This was carried out according to the following procedure:

50 mg of hydrogel was swollen in 15 mL of deionised water for 30 minutes. The swollen hydrogel was then forced through a 21 G graded needle (0.8 mm ID, 40 mm length) into a suitable container comprising a 125 mL propylene pot (Fisher), which action disrupted the gel into tiny pieces. The syringe body was then flushed over the container with two 10 mL aliquots of deionised water. The volume in the container was made up to 50 mL with deionised water. The following solutions were then added to this disintegrated gel suspension:
10 mL of reagent mix (5 parts of 0.1 M sodium phosphate (Fisher), pH 6+4 parts 2% w/w starch (soluble; Sigma)+1 part of 1 mg/mL lactoperoxidase enzyme 1050 U/mg; DMV International, Holland;
5 mL of 100 mM potassium iodide (Fisher); and
5 mL of 40% w/w glucose (Fisher) solution.

These were mixed together quickly. Time=0 was counted from the addition of the glucose. After 5 minutes, 1 mL of 5 M HCl was added to stop the reaction. The absorbance was then read at 630 nm using a UV-VIS spectrophotometer (supplier: CamSpec, type: M302). If the colour intensity was too great to allow an accurate reading, the sample was diluted with a defined volume of deionised water to bring the colour back on scale. The results were expressed as percentage recovery, by reference to the absorbance measured in the pre-gamma irradiation hydrogel samples.

### EXAMPLE 1

### Extremely Low Enzyme Activity Recovery

Nine hydrogels based on the core ingredients of Table 1 were prepared, irradiated and assayed using the procedures described above. Eight of the hydrogels each included, in addition to the core ingredients, one ingredient indicated in the literature (see, for example, Ahmad A., Akhtar M. S. and Bhakuni (2001) Biochemistry, 40(7), 1945-1955; Akhtar M. S., Ahmad A. and Bhakuni (2002) Biochemistry, 41(22), 7142-7149; Gouda M. D., Singh S. A., Rao A. G. T., Thakur M. S. and Karanth N. G. (2003) Journal of Biological Chemistry, 278(27), 24324-24333; and Eremin A. N., Metelitsa D. I., Shishko Z. F., Mikhailova R. V., Yasenko M. I. and Lobanok A. G. (2001) Applied Biochemistry and Microbiology, 37(6), 578-586) to have a stabilising effect on glucose oxidase or other enzymes against heat or chemical degradation. The ingredients were as follows; magnesium chloride, sodium chloride, potassium chloride, calcium chloride, potassium phosphate, glycerol, PVA (MW range 31,000 to 50,000) and PVA (MW range 89,000 to 99,000). One hydrogel containing the core ingredients only (i.e. without any additives) was prepared as a control.

The results are shown in Table 2 below.

TABLE 2
Effect of additives on the recovery of glucose oxidase activity in
poly-AMPS based hydrogels following sterilisation by
gamma irradiation. Pre-gamma activity = 100%.

| | Recovery of glucose |
|---|---|
| Additives | oxidase activity |
| No additives | <1% |
| Magnesium chloride (4%) | <1% |
| Sodium chloride (4%) | <1% |
| Potassium chloride (4%) | <1% |
| Calcium chloride (5%) | <1% |
| Potassium phosphate (2%) | <1% |
| Glycerol (10%) | <1% |
| PVA, either 31,000-50,000 or | <1% |
| 89,000-98,000 MW (1%) | |

It will be seen from the above table that the activity of glucose oxidase in the control hydrogel was almost completely destroyed during gamma irradiation. Further, the range of compounds suggested in the literature as stabilisers of glucose oxidase or other enzymes against heat or chemical degradation offered little or no protection of the enzyme during gamma irradiation. The stabilisers tested did not improve the recovery of glucose oxidase activity.

### EXAMPLE 2

### Slight Recovery of Enzyme Activity

Some ingredients were found to exhibit a slight protective effect on glucose oxidase during gamma irradiation.

Three hydrogels containing the core ingredients of Table 1 and either ammonium sulphate (4%), gelatin (1%) or ammonium AMPS (15%) were prepared, irradiated and assayed as described above.

The results are shown in Table 3 below.

TABLE 3
Effect of additives on the recovery of glucose oxidase activity
in poly-AMPS based hydrogels following sterilisation by
gamma rays. Pre-gamma activity = 100%.

| Additives | Recovery of glucose oxidase activity |
|---|---|
| Ammonium sulphate (4%)* | 4.3% |
| Ammonium (from 15% ammonium | 3.9% |
| AMPS/15% Na AMPS)** | |
| Gelatin (1%) | 3.4% |

*This corresponds to the total concentration of approximately 1.1% of ammonium cation NH₄⁺.
**This corresponds to the total concentration of approximately 1.2% of ammonium cation NH₄⁺.

It will be seen from the above that the presence of ammonium ions (either from ammonium sulphate or from ammonium AMPS as the main gel component) and/or gelatin facilitates a slight recovery of glucose oxidase activity.

### EXAMPLE 3

### Satisfactory Recovery of Enzyme Activity

Five hydrogels containing the core ingredients of Table 1 and either zinc L-lactate (0.2% or 1.0%), zinc chloride (1.0%), zinc sulphate (1.0%) or sodium lactate (1.0%) were prepared, irradiated and assayed as described above.

The results are shown in Table 4 below.

TABLE 4
Effect of additives on the recovery of glucose oxidase activity
in poly-AMPS-based hydrogels following sterilisation by
gamma irradiation. Pre-gamma activity = 100%.

| | Recovery of glucose |
|---|---|
| Additives | oxidase activity |
| Zinc L-lactate (0.2%) | 8.1% |
| Zinc L-lactate (1%) | 24.9% |
| Zinc chloride (1%) | <1% |
| Zinc sulphate (1%) | <1% |
| Sodium lactate(1%) | <1% |

It will be seen from the above that zinc lactate was found to provide a considerable protective effect on glucose oxidase during gamma irradiation. The magnitude of the effect was found to increase with increasing amount of zinc L-lactate. The protective effect appeared to require the presence of both zinc cation and lactate anion. Using other zinc salts, (e.g. chloride or sulphate) or other lactate compounds (e.g. sodium lactate) did not give a desirable recovery of glucose oxidase activity under these conditions.

### EXAMPLE 4

### Very Good Recovery of Enzyme Activity

The following example tested the protective effect of zinc L-lactate in the presence of additional ingredients.

Five hydrogels containing the core ingredients of Table 1 and zinc L-lactate (0.2% or 1.0%) and either PVA (MW 89,000-98,000) (1%), ammonium sulphate (10% or 20%), gelatin (4%) or ammonium AMPS (15%) were prepared, irradiated and assayed as described above.

The results are shown in Table 5 below.

TABLE 5
Effect of additives on the recovery of glucose oxidase activity
in poly-AMPS based hydrogels following sterilisation by
gamma irradiation. Pre-gamma activity = 100%.

| | Recovery of glucose |
|---|---|
| Additives | oxidase activity |
| Zinc L-lactate (0.2%) + PVA 90,000 MW (1%) | 15.2% |
| Zinc L-lactate (1%) + Ammonium sulphate (10%) | 33.6% |
| Zinc L-lactate (1%) + Ammonium sulphate (20%) | 34.9% |
| Zinc L-lactate (1%) + Gelatin (4%) | 51.2% |
| Zinc L-lactate (1%) + Ammonium (from 15% | 52.7% |
| ammonium AMPS/15% Na AMPS)* | |
| Sodium D, L-lactate (1%) + Ammonium (from | 61.4% |
| 15% ammonium AMPS)* | |
| Sodium L-lactate (1%) + Ammonium (from 15% | 60.7% |
| ammonium AMPS)* | |
| Sodium D-lactate (1%) + Ammonium (from 15% | 64.3% |
| ammonium AMPS)* | |

*This corresponds to the total concentration of approximately 1.2% of ammonium cation NH₄⁺.

It will be seen from the above that more than 50% recovery of glucose oxidase activity was achieved using either the combination of zinc L-lactate and gelatin or the combination of zinc L-lactate and ammonium AMPS. Similarly, the combination of sodium lactate (in D-, L- or D, L-form) and ammonium AMPS resulted in more than 50% recovery of glucose oxidase. The combined effect of zinc L-lactate and ammonium sulphate resulted in the recovery of approximately 34% of the glucose oxidase activity. PVA was also found to augment the protective effect of 0.2% zinc L-lactate (but the augmentation was not evident at higher zinc L-lactate levels).

### EXAMPLE 6

The ensuing composition in accordance with the invention is a skin treatment product of the form shown in FIG. 6 of WO 03/090800, which comprises a glucose-containing hydrogel slab as a lower layer of the product, and an upper layer comprising a poly-AMPS hydrogel that incorporates glucose oxidase.

The hydrogel lower layer was formulated to include the following ingredients by weight:

| | |
|---|---|
| Water (ex Fisher, distilled, de-ionised, analytical grade) | 54.7% |
| Sodium AMPS (ex Lubrizol AMPS 2405 Monomer) | 40.0% |
| Polyethylene glycol diacrylate (PEG400 diacrylate, ex UCB | 0.19% |
| Chemicals available as Ebecryl 11) | |
| 1-hydroxycyclohexyl phenyl ketone (a photoinitiator, ex | 0.01% |
| Aldrich) | |
| Anhydrous glucose (enzyme substrate, ex Fisher) | 5.00% |
| Potassium iodide (ex Fisher) | 0.05% |
| Zinc L-lactate hydrate (ex Aldrich) | 0.10% |

The mixture was dispensed into casting trays containing either polyester scrim (polyester non-woven, open mesh support, available from HDK Industries Inc, Product Code 5722) or polyethylene net support, of dimensions 100 mm×100 mm, to a depth of about 1.5 mm. The polyethylene net support was fabricated from polyester staple fibres thermally bonded by a polyester resin—Product code 5722, from Castle Industries, Greenville, S.C. 9609, USA. The hydrogel was then set, by irradiation under a UV lamp, for up to 60 seconds and a power rating of approximately 100 mW/cm². The hydrogel was then allowed to cool to 30° C. or below.

The enzyme-containing hydrogel was formulated to include the following ingredients by weight:

| | |
|---|---|
| Water (ex Fisher, distilled, de-ionised, analytical grade) | 58.6% |
| Sodium AMPS (ex Lubrizol AMPS 2405 Monomer) | 20.0% |
| Ammonium AMPS (ex Lubrizol AMPS 2411 Monomer) | 20.0% |
| Polyethylene glycol diacrylate (PEG400 diacrylate, ex UCB | 0.19% |
| Chemicals available as Ebecryl 11) | |
| 1-hydroxycyclohexyl phenyl ketone (a photoinitiator, ex | 0.01% |
| Aldrich) | |
| Glucose oxidase (GOX, Biocatalysts, Pontypridd, Code | 0.035% |
| G575P) | |
| Zinc L-lactate hydrate (ex Aldrich) | 1.0% |
| Pluronic P65 (block co-polymer of ethylene oxide and | 0.15% |
| propylene oxide, | |
| HO—[CH2CH2O]x—[CH2CHCH3O]y—[CH2CH2O]y—H, | |
| average MW 3400 (BASF) | |

The mixture was dispensed into casting trays containing polyester scrim (polyester non-woven, open mesh support, available from HDK Industries Inc, Product Code 5722) of dimensions 100 mm×100 mm, to a depth of about 1.0 mm. The hydrogel was then set, by irradiation under a UV lamp, for up to 30 seconds (typically 25 seconds), and a power rating of approximately 100 mW/cm². The hydrogel was then allowed to cool to 30° C. or below.

The enzyme-containing hydrogel and the glucose-containing hydrogel were brought together, one overlying the other.

An oxygen-permeable and moisture-permeable covering or overlay such as of polyurethane may be located over the enzyme-containing hydrogel and may be adhered to the skin by means of e.g. acrylic adhesive provided on the lower face of the overlay.

The resulting product was packaged in an oxygen-impermeable pouch or enclosure, e.g. made of laminated aluminium foil pouches as supplied by Sigma (code Z183407).

### EXAMPLE 7

### Satisfactory Recovery of Catalase Activity in an Aqueous Sample in the Presence of Zinc Lactate

Experiment

Overall Experimental Plan

Two aqueous solutions of catalase (1 mg ml⁻¹) were prepared. The catalase used was as follows: enzyme classification number EC 1.11.1.6. from Sigma, catalogue no. C9322, 2,380 Units per mg. One of the solutions contained zinc L-lactate (1% w/w), the other solution did not contain any additives. The solutions were next irradiated with gamma rays by an industry-standard sterilizing service, with a dose range as normally specified for hydrogel wound dressings. The solutions were then analysed for remaining enzyme activity.

Catalase Activity Assay

The solutions, both pre- and post-gamma irradiated, were assayed for catalase activity. This was performed according to the following procedure:

50 μL of the aqueous sample was added into 50 mL of phosphate buffer (50 mM). Hydrogen peroxide (500 μL, 30% w/w—Sigma) was added and the mixture was incubated while shaking moderately for 20 minutes. The following solutions were then added to the mixture:
5 mL of reagent mix (5 parts of 0.1 M sodium phosphate, pH 6+4 parts 2% w/w starch+1 part of 1 mg/mL lactoperoxidase enzyme)
5 mL of 100 mM potassium iodide

Time=0 was counted from the addition of the potassium iodide. After 5 min, the absorbance was read at 630 μm. If the colour intensity was too great to allow an accurate reading, the sample was diluted with a defined volume of deionised water to bring the colour back on scale. The intensity of the colour was indirectly proportional to the catalase activity in the sample. The results were expressed as percentage recovery, by reference to the absorbance measured in the pre-gamma irradiation samples.

Result

Zinc lactate was found to provide a considerable protective effect on catalase during the gamma treatment (Table 6). Whilst no catalase activity was recovered after gamma treatment in aqueous solution in the absence of zinc lactate there was a considerable recovery of catalase activity in the presence of zinc lactate.

TABLE 6
Effect of additives on the recovery of catalase activity in aqueous solution
following sterilisation by gamma irradiation. Pre-gamma activity = 100%.

| | Recovery of |
|---|---|
| Additives | catalase activity |
| No additives (i.e. water only) | <1% |
| Zinc L-lactate (1%) | 83.5% |

### EXAMPLE 8

### Satisfactory Recovery of Lactoperoxidase Activity in Poly-AMPS Hydrogel in the Presence of Zinc Lactate

Experiment

Overall Experimental Plan

Poly-AMPS hydrogels containing lactoperoxidase (1 mg of enzyme per gram of gel) were prepared using the procedure set out for glucose oxidase. The lactoperoxidase used was as follows: enzyme classification number EC 1.11.1.7. from DMV International, Holland, 1050 Units per mg. The gels were next irradiated with gamma rays by an industry-standard sterilizing service, with a dose range as normally specified for hydrogel wound dressings constructed from this polymer. The solutions were then analysed for remaining enzyme activity.

Lactoperoxidase Activity Assay

The gel slabs, both pre- and post-gamma irradiated, were assayed for lactoperoxidase activity. This was performed according to the following procedure:

50 mg of gel was swollen in 15 mL of deionised water for 30 mins. The gel was then forced through a 21 G graded needle (0.8 mm ID, 40 mm length). This effectively disrupted the gel into tiny pieces. The syringe body was flushed with two 5 mL aliquots of deionised water. The volume was made up to 50 mL with phosphate (Fisher)/citrate (Fisher) buffer (pH 5). 2.5 mL aliquot of this gel suspension was taken and the following solutions were added:
2 μL of hydrogen peroxide (30% w/w—Sigma)
50 μL of 3,3′,5,5′-tetramethylbenzidine (Sigma) (1 mg dissolved in 1 mL of dimethylsulphoxide (Sigma))

Time=0 was counted from the addition of the potassium iodide. After 5 min, the absorbance was read at 630 nm. If the colour intensity was too great to allow an accurate reading, the sample was diluted with a defined volume of deionised water to bring the colour back on scale. The intensity of the colour was directly proportional to the lactoperoxidase activity in the sample. The results were expressed as percentage recovery, by reference to the absorbance measured in the pre-gamma irradiation samples.

Result

Zinc lactate was found to provide a considerable protective effect on lactoperoxidase during the gamma treatment (Table 7). The effect was only studied in ammonium AMPS based gels. Whilst 27.2% lactoperoxidase activity was recovered after gamma treatment in the ammonium AMPS-based gel in the absence of zinc lactate there was more than a 3-fold improvement in the enzyme activity retained in the presence of zinc lactate.

TABLE 7
Effect of additives on the recovery of lactoperoxidase activity
in poly-AMPS based hydrogels following sterilisation by
gamma irradiation. Pre-gamma activity = 100%.

| | Recovery |
|---|---|
| Additives | of lactoperoxidase activity |
| Ammonium (from 50% ammonium AMPS)* | 27.2% |
| Zinc L-lactate (1%) + Ammonium (from | 95.5% |
| 15% ammonium AMPS)* | |

*This corresponds to the total concentration of approximately 1.2% of ammonium cation NH₄⁺.

1. A composition comprising an enzyme, a source of lactate ions and a source of zinc ions and/or a source of ammonium ions.

2. A composition according to claim 1, wherein the enzyme is in hydrated condition.

3. A composition according to claim 1 or 2, wherein the source of ammonium ions comprises ammonium sulphate or 2-acrylamido-2-methyl propanesulphonic acid, ammonium salt (ammonium AMPS).

4. A composition according to claim 1, wherein the source of zinc ions is any compound capable of releasing zinc ions or zinc-containing ions in water.

5. A composition according to claim 4, wherein the source of lactate ions is any compound capable of releasing lactate ions or lactate-containing ions in water.

6. A composition according to claim 5, wherein the source of zinc ions and source of lactate ions is zinc lactate.

7. A composition according to claim 6, wherein the source of zinc ions and source of lactate ions is zinc L-lactate.

8. A composition according to claim 1, wherein the composition additionally comprises one or more ingredients selected from sugar alcohols, proteins and neutral water-soluble polymers.

9. A composition according to claim 8, wherein the composition additionally comprises a source of proteins.

10. A composition according to claim 1, wherein the composition is sterilised by irradiating the composition with sterilising radiation.

11. A composition according to claim 10, wherein the sterilising radiation is gamma radiation.

12. A composition according to claim 1, wherein the enzyme comprises an oxidase.

13. A composition according to claim 12, wherein the oxidase comprises glucose oxidase.

14. A composition according to claim 12 or 13, including zinc lactate.

15. A composition according to claim 12 or 13, including sodium lactate and ammonium AMPS.

16. A composition according to claim 1, wherein the enzyme comprises catalase.

17. A composition according to claim 1, wherein the enzyme comprises lactoperoxidase.

18. A method of stabilising an enzyme in a composition during exposure to sterilising radiation by bringing the enzyme into contact with a source of zinc ions and/or a source of ammonium ions and a source of lactate ions.

19. A product comprising a composition in accordance with claim 1.

20. A product according to claim 19, wherein the product is a skin treatment product and the enzyme is an oxidase.

21. A product according to claim 20, wherein the skin treatment product is a skin dressing.

22. A product according to claim 21, wherein the dressing incudes one or more hydrated hydrogels.

23. A product according to claim 22, wherein the oxidase enzyme, source of zinc ions and/or a source of ammonium ions and source of lactate ions are present in one or more hydrated hydrogels.

24. A product according to claim 20, including a source of substrate for the oxidase enzyme.

25. A product according to claim 24, wherein the substrate is located in a hydrated hydrogel.
